(19) United States
(12) Patent Application Publication
(10) Pub. No.: US 2023/0020979 A1
(43) Pub. Date: Jan. 19, 2023

(54) DEVICE FOR DETECTING PRESENCE OR ABSENCE OF A CHEMICAL OR BIOLOGICAL TARGET WITHIN A SAMPLE COMPRISING AN ELECTRODE FUNCTIONALISED WITH AN ANTIBODY AND CORRESPONDING DETECTION METHOD

(71) Applicant: OSLER DIAGNOSTICS LIMITED, Oxford (GB)

(72) Inventor: Samuel Whitby, Oxford (GB)

(73) Assignee: OSLER DIAGNOSTICS LIMITED, Oxford (GB)

(21) Appl. No.: 17/779,672

(22) PCT Filed: Nov. 27, 2020
(86) PCT No.: PCT/GB2020/053033

§ 371 Date: May 25, 2022

(30) Foreign Application Priority Data
Nov. 28, 2019 (GB) 1917348.3
Nov. 28, 2019 (GB) 1917351.7
Nov. 28, 2019 (GB) 1917356.6

(51) Int. Cl. G01N 27/327; G01N 27/416; G01N 33/543; G01N 33/68; B01L 3/00

(52) U.S. Cl. CPC G01N 27/3276; G01N 27/3277; G01N 27/416; G01N 33/5438; G01N 33/68; B01L 3/502761; G01N 2333/4737; B01L 2200/0684; B01L 2200/0652; B01L 2300/0645; B01L 2200/16; B01L 2400/0487; B01L 2200/04

(57) ABSTRACT

A device, liquid handling cartridge and related method for detecting the presence or absence of a chemical or biological target within a sample. The method includes the steps of: providing an electrochemical cell with a first electrode module and a second electrode; providing an electronic component between the first electrode module and the second electrode; introducing the sample into the electrochemical cell; measuring the potential difference between the first electrode module and second electrode; and confirming the presence of the chemical or biological target if the measured potential difference exceeds a predetermined threshold value.

### PRIORITY CLAIM

The present application is a National Phase entry of PCT Application No. PCT/GB2020/053033, filed Nov. 27, 2020, which claims priority from GB Application No. 1917348.3, filed Nov. 28, 2019, GB Application No. 1917351.7, filed Nov. 28, 2019, and GB Application No. 1917356.6, filed Nov. 28, 2019, all of these disclosures being hereby incorporated by reference in their entirety.

### FIELD OF THE INVENTION

The present invention relates to improvements in or relating to a method for sensing a chemical or biological target and in particular, a method for sensing the formation or depletion of this target.

### BACKGROUND OF THE INVENTION

Electrochemical techniques such as electrochemical impedance spectroscopy (EIS) can be an effective technique for probing the features of surface-modified electrodes. EIS is capable of sensitively monitoring the changes in capacitance or charge-transfer resistance associated with the specific binding of certain materials to a suitable electrode surface. Other techniques such as cyclic voltammetry (CV), pulsed voltammetry methods for example, differential pulsed voltammetry (DPV) and square wave voltammetry (SWV), may also be used to probe the surface of the modified electrodes. In addition, features of the surface-modified electrodes may be measured chronoamperometrically i.e. by measuring a current through time. Alternatively, the features of the surface-modified electrodes may be measured potentiometrically, either at the open circuit (OCP) or with the application of a fixed current (chronopotentiometrically).

In some instances, electrochemical measurements may utilise a three way electrode cell. The three way electrode cell may comprise a reference electrode, a counter electrode and a working electrode, whereby a current flowing between the counter electrode and the working electrode can be continuously monitored.

Real-time electrochemical measurement of a chemical or biological target that is being generated in a redox active solution is not achievable by most standard techniques because often the solution is redox active in the electrolyte solution across the part of the potential window needed to detect the electroactive species. A special case of electroactive species are precipitates that contain both oxidised and reduced members of the redox couple. These poise the open circuit potential (OCP) of the electrode to their half-wave potential (E_{1/2} vs reference).

Measuring OCP over time for these systems can yield information about the development of the precipitate without the application of a current. However, OCP is unreliable as a sensing technique for various reasons. Firstly, it is poorly defined for systems that do not contain appreciable concentrations of a reversible redox couple, so there is no reliable baseline for negative control, leading to false positives. Secondly, small perturbations at the sensor or sample can cause large changes to OCP, taking many minutes to re-equilibrate.

Additionally, the magnitude of OCP is insensitive to the quantity of electroactive species, only providing a binary, rather than quantitative result.

Additionally, measurement of OCP requires electrometers with very high input impedance in the range of tera Ohms, in order to not produce artefacts. These are expensive so are not applicable to all point of care applications.

Therefore, there is a requirement for a modified potentiometric technique that is capable of utilising hardware that is suitable for point of care applications in order to provide a simple and efficient technique for quantitatively measuring chemical or biological targets which may be electroactive species. In addition, it is also highly desirable to provide a modified potentiometric technique for predicting behaviour in the absence of the target, which may be an electroactive species, fast equilibration of signal, and quantitative detection of the target. Moreover, it would be desirable to provide a technique that can also be applied to any electroactive species in solution as well as surface-bound.

It is against this background that the present invention has arisen.

### SUMMARY OF THE INVENTION

According to the present invention, there is provided a method for detecting the presence or absence of a chemical or biological target within a sample, the method comprising the steps of:

providing an electrochemical cell with a first electrode module and a second electrode;

providing an electronic component between the first electrode module and the second electrode;

functionalising the second electrode with an antibody; introducing the sample into the electrochemical cell;

measuring the potential difference between the first electrode module and second electrode; and

confirming the presence of the chemical or biological target if the measured potential difference exceeds a predetermined threshold value.

The method may further comprise the step of varying the resistance of the electronic component. The degree to which the resistor modifies the potential difference can be tuned by varying its resistance. In some embodiments, the resistance can be at zero, the electrochemical cell may be short circuited so the measured potential can become zero. As the resistance approaches infinity, the potential difference may tend to the OCP value. The magnitude of the resistor may be varied during measurements in order to optimise performance.

For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

In some embodiments, an example of a sandwich-format immunoassay can be detecting a binding event between an immobilised primary antibody and a CRP-secondary antibody-HRP complex using an electrochemical measurement technique. A sample containing CRP can be incubated with a secondary antibody and an enzyme label in a single solution. The primary antibody can be immobilised on an electrode surface, such as the second electrode (working electrode). The immobilised primary antibody can then be contacted with the single solution containing the target analyte, the secondary antibody and the enzyme label to create a sandwich complex bound to an electrode surface, such as the second electrode (working electrode). The sandwich complex can then be in contact with a substrate for the HRP, wherein the substrate is converted by the enzyme into an insoluble precipitate on the electrode surface. An electrochemical signal can be measured during and/or after the formation of the insoluble precipitate on the electrode surface.

In some embodiments, the method may further comprise the step of varying the resistance of the electronic component.

In some embodiments, the chemical or biological target is a redox poising species.

In some embodiments, the method may further comprise the step of providing a component configured to bind to the first electrode module to generate or change the concentration of the chemical or biological target on the first electrode module.

According to an aspect of the invention, there is provided a device for detecting the presence or absence of a chemical or biological target within a sample, the device comprising:

an electrochemical cell comprising a first electrode module, a second electrode and

an electrolyte container configured to immerse both the first electrode module and

the second electrode in an electrolyte solution, wherein the second electrode is functionalised with an antibody;

an electronic component provided between the first electrode module and the second electrode;

an electrometer configured to measure the potential difference between the first electrode module and the second electrode;

a memory configured to store predetermined threshold data; and

a processor configured to compare the measures potential difference and the stored threshold data to confirm the presence or absence of the chemical or biological target within the sample.

In some embodiments, the electronic component is a variable resistor including either a rheostat or varistor or a switched group of resistors.

In some embodiments, the memory further comprises data for different values of the resistance of the electronic component.

In some embodiments, the second electrode is functionalised with a biological recognition element.

In some embodiments, the second electrode is made from gold.

In some embodiments, the first electrode module is provided with a component configured to generate or change the concentration of the chemical or biological target.

In some embodiments, the first electrode module comprising the component is configured to generate a different concentration of the chemical or biological target from the concentration of the chemical or biological target on the second electrode.

In some embodiments, the first electrode module comprises a single first electrode.

In some embodiments, the first electrode is made from silver.

In some embodiments, the first electrode is coated in silver chloride.

In some embodiments, the first electrode module comprises two electrodes, one connected to the electrometer and one connected to the electronic component.

According to an aspect of the present invention, there is provided a liquid handling cartridge for detecting the presence or absence of a chemical or biological target within a sample, the cartridge comprising one or more measurement chambers comprising a device as described above for performing measurements on the sample.

In some embodiments, the liquid handling cartridge further comprising: a main chamber;

a sample chamber for receiving a sample;

a variable pressure source conduit for connecting the main chamber to a variable pressure source;

a sample chamber conduit which fluidically connects the sample chamber to the main chamber;

a sample chamber conduit valve for opening and closing the sample chamber conduit;

a respective measurement chamber conduit for each measurement chamber, wherein each respective measurement chamber conduit fluidically connects the respective measurement chamber to the main chamber; and

a respective measurement chamber conduit valve for opening and closing each respective measurement chamber conduit.

The variable pressure source conduit may be connected to the main chamber at the top of the main chamber, to reduce the likelihood of liquids in the main chamber entering the variable pressure source conduit and variable pressure source.

The main chamber and/or one or more of the one or more measurement chambers may comprise a reagent, as described below.

The liquid handling device may be for performing a diagnostic test on a sample.

The liquid handling device allows a sample to be transferred from the sample chamber into the main chamber by reducing the pressure in the main chamber relative to the sample chamber. Precise control of the volume of sample transferred into the main chamber is possible by controlling the pressure change in the main chamber. In the main chamber, the sample may react or mix with a reagent. The device allows the sample to be held in the main chamber for as long as necessary, for example for a duration of time needed to complete a reaction with a reagent. This may not be readily achievable with known fluid handling devices, such as conventional microfluidic devices.

The sample may then be transferred from the main chamber to the measurement chamber where it is held while a measurement is performed, for example as part of a diagnostic test such as an immunoassay. Again, precise control of the volume of sample transferred into the measurement chamber and residence time in the measurement chamber are possible.

The liquid handling device may be provided with or without a variable pressure source.

That is to say that a variable pressure source may be integrated into the liquid handling device, but is preferably reversibly connected to the liquid handling device and as such may be provided separately.

A variable pressure source is a pressure source that can apply or provide both positive and negative pressure changes. For example, the variable pressure source may be a syringe and may be controlled by a stepper motor. Other variable pressure sources and ways of controlling variable pressure sources are known to the skilled person.

The liquid handling device is not limited to having only one main chamber or only one variable pressure source.

The main chamber may be arranged to receive a fluid from the sample chamber when the sample chamber conduit valve is open and a negative pressure change is applied to the main chamber via the variable pressure source conduit, and the one or more measurement chambers are arranged to receive the fluid from the main chamber when the respective measurement chamber conduit valves are open and a positive pressure change is applied to the main chamber via the variable pressure source conduit.

The liquid handling device may further comprise one or more reagent chambers; a respective reagent chamber conduit for each reagent chamber, wherein the respective reagent chamber conduit fluidically connects the respective reagent chamber to the main chamber; and a respective reagent chamber conduit valve for opening and closing each respective reagent chamber conduit.

The reagent chambers may store reagents such as an antibody or protein solution, antibody or protein powder, buffer solution, an enzyme substrate such as 3,3′,5,5′-tetramethylbenzidine “TMB,” and so on, for mixing or reacting with the sample in order to facilitate a measurement on the sample in the measurement chamber, for example to perform a diagnostic test on the sample.

The reagents in the reagent chambers may be readily mixed with the sample by controlling pressure changes in the liquid handling device. By providing a main chamber surrounded by one or more reagent chambers, the device facilitates complex mixing operations, for example operations with multiple steps each requiring precise volume control and timing that may not be readily achieved using known fluid handling devices.

A reagent chamber may also be known as an assay chamber.

The one or more measurement chambers may comprise a first measurement chamber for performing a first measurement on the sample and a second measurement chamber for performing a second measurement on the sample. As such, the liquid handling device further comprises a first respective measurement chamber conduit which fluidically connects the first measurement chamber to the main chamber; a second respective measurement chamber conduit which fluidically connects the second measurement chamber to the main chamber; a first respective measurement chamber 5 conduit valve for opening and closing the first respective measurement chamber conduit; and a second respective measurement chamber conduit valve for opening and closing the second respective measurement chamber conduit.

As such, a single liquid handling device may be configured to receive only one sample in the sample chamber yet perform multiple measurements or diagnostic tests for determining multiple properties of the sample.

The one or more reagent chambers may comprise one or more first dedicated reagent chambers for reagents to be used only in a diagnostic test to be performed in the first measurement chamber, one or more second dedicated reagent chambers for reagents to be used only in a diagnostic test to be performed in the second measurement chamber, and one or more shared reagent chambers for reagents to be used in the diagnostic tests to be measured in both the first and second measurement chambers.

Ordinarily, separate measurement chambers would each require their own separate reagent sources, however, by providing a shared reagent chamber that provides a reagent, such as a buffer solution, common to two separate diagnostic tests or measurements, a more compact liquid handling device may be provided. At the same dedicated reagent chambers store reagents, such as specific antibodies or proteins that may be selectively mixed with the sample for particular diagnostic tests or measurements providing the device with a broader range of functionality.

The liquid handling device comprising one or more reagent chambers may further comprise a mixing chamber for mixing the sample with a reagent from one of the one or more reagent chambers. As such, the device also comprises a mixing chamber conduit, wherein the mixing chamber conduit fluidically connects the mixing chamber to the main chamber; and a mixing chamber conduit valve for opening and closing the mixing chamber conduit.

Once a reagent is combined with the sample, the resulting combination may be shuttled (transferred back and forth) between the main chamber and mixing chamber to accelerate mixing of the reagent and sample (homogenise the reagent and sample) or accelerate dissolution of the reagent in the sample or other liquid.

The liquid handling device may further comprise a waste chamber and a waste chamber conduit, wherein the waste chamber conduit fluidically connects 5 the waste chamber to the main chamber.

The waste chamber may be used to safely store excess sample and/or reagents, for example after the liquid handling device has been used to perform a measurement on the sample. Further, sample may be overprovided to the main chamber, and then transferred into another chamber such as a mixing chamber in a precise quantity, while the excess sample is expelled to the waste chamber. The precisely measured sample can then be returned to the main chamber with a precise known volume.

The liquid handling device may further comprise a waste chamber conduit valve for opening and closing the waste chamber conduit. Alternatively, the waste chamber conduit may fluidically connect the waste chamber to the main chamber via the measurement chamber. Thus, sample can be transferred directly from the measurement chamber to the waste chamber after a measurement has been performed.

At least one of the one or more measurement chambers may comprise a plurality of electrodes. The plurality of electrodes may be for performing an electrochemical measurement. Alternatively or in addition, at least one of the one of more measurement chambers may comprise an element for performing an optical measurement, such as a window. In general, each measurement chamber may be configured for any type of measurement: electrochemical, optical (photometry, fluorescence, imaging), magnetic (magnetic field sensor) and/or thermometric (temperature sensor).

Each conduit valve may be a pinch valve. A pinch valve may be operated by an external actuator that selectively applies pressure to the pinch valve to open or close it. Optionally, the conduit valves may be configured in a circular array, so that they can be operated by an actuator with a circular array of actuation elements. A pinch valve is a valve which uses a pinching effect to obstruct fluid flow.

The conduit valves of the devices described above may be configured such that only one valve is open at any given time. The conduit valves of the devices described above may be closed by default.

The chambers of the liquid handling device may comprise gas exchange means, such as holes, air vents or a breathable sealing film. A breathable sealing film may be a hydrophobic porous sealing film with medical-grade adhesive, such as those that are available from AeraSeal® or Breathe-Easy® for cell and tissue culture where gas exchange is required. The gas exchange means is for allowing air or any other ambient gas to enter and exit each chamber to balance a pressure change resulting from liquid (such as a sample or reagent) entering the respective chambers, although this is not essential. Depending on the chamber, the chamber may be opened to the air (for example have an unsealed top), the chamber may have an air vent open to the outside, a fluid port or channel may act as an air vent, or the chamber could be vented via a venting valve connected to the pressure source conduit (for example, in the case of a main chamber).

The liquid handling device may be made from conventional materials known to the skilled person such as glass, silicon, polydimethylsiloxane (PDMS) or any thermoplastic (such as polymethylmethacrylate (PMMA), polycarbonate (PC), cyclic olefin copolymer (COC), polypropylene (PP)) using conventional methods such as chemical etching, laser etching, routing or moulding.

The present invention is further described in GB2004723.9, the contents of which are hereby incorporated by reference in their entirety.

According an aspect of the present invention there is provided a method for detecting the presence or absence of a chemical or biological target within a sample, the method comprising the steps of:

providing an electrochemical cell with a first electrode module and a second electrode;

providing an electronic component between the first electrode module and the second electrode;

introducing the sample into the electrochemical cell;

measuring the potential difference between the first electrode module and second electrode;

confirming the presence of the chemical or biological target if the measured potential difference exceeds a predetermined threshold value.

The method may further comprise the step of varying the resistance of the electronic component. The degree to which the resistor modifies the potential difference can be tuned by varying its resistance. In some embodiments, the resistance can be at zero, the electrochemical cell may be short circuited so the measured potential can become zero. As the resistance approaches infinity, the potential difference may tend to the OCP value. The magnitude of the resistor may be varied during measurements in order to optimise performance.

The method may further comprise the step of functionalising the second electrode with a biological recognition element such as an antibody. For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

In some embodiments, an example of a sandwich-format immunoassay can be detecting a binding event between an immobilised primary antibody and a CRP-secondary antibody-HRP complex using an electrochemical measurement technique. A sample containing CRP can be incubated with a secondary antibody and an enzyme label in a single solution. The primary antibody can be immobilised on an electrode surface, such as the second electrode (working electrode). The immobilised primary antibody can then be contacted with the single solution containing the target analyte, the secondary antibody and the enzyme label to create a sandwich complex bound to an electrode surface, such as the second electrode (working electrode). The sandwich complex can then be in contact with a substrate for the HRP, wherein the substrate is converted by the enzyme into an insoluble precipitate on the electrode surface. An electrochemical signal can be measured during and/or after the formation of the insoluble precipitate on the electrode surface.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

The chemical or biological target may be a redox poising species.

The method may further comprise the step of providing a component configured to bind to the first electrode module to generate or change the concentration of the chemical or biological target on the first electrode module.

For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

Furthermore, according to the present invention there is provided a device for detecting the presence or absence of a chemical or biological target within a sample, the device comprising:

an electrochemical cell comprising a first electrode module, a second electrode and an electrolyte container configured to immerse both the first electrode module and the second electrode in an electrolyte solution;

an electronic component provided between the first electrode module and the second electrode;

an electrometer configured to measure the potential difference between the first electrode module and the second electrode;

a memory configured to store predetermined threshold data; and

a processor configured to compare the measures potential difference and the stored threshold data to confirm the presence or absence of the chemical or biological target within the sample.

The electronic component may be a variable resistor including either a rheostat or varistor or a switched group of resistors.

Alternatively, the electronic component could also be a diode or a transistor. The memory may further comprise data for different values of the resistance of the electronic component. The data storage may be in any suitable form of memory, either volatile or non-volatile, including but not limited to RAM, SRAM, ROM, EEPROM or Flash memory. The conversion data stored within the data storage comprises a set of calibration values correlating the potential difference measurements when known number of moles of a given electroactive species have been formed or depleted. This data may be retained in the form of individual data points or it may be processed to provide a functional description of the inter-relation between the potential difference and the number of moles of the electroactive species that are formed or depleted. The conversion data may be provided as a closed set of data with which all future potential difference measurements are compared. Alternatively, the data set may be augmented or replaced by periodic calibration of the device.

The second electrode may functionalised with a biological recognition element, which may be an antibody. Antibodies, including antibody fragments, suitable for use in the methods described herein are known in the art. For example, the antibody or the antibody fragment may be selected from one or more of the classes IgA, IgD, IgE, IgG and IgM. In some embodiments, the antibody or antibody fragment is of the IgG type. The antibody binds selectively to a target species or target analyte. The antibody or antibody fragment may be derived from a mammal, including, but not limited to, a mammal selected from a human, a mouse, a rat, a rabbit, a goat, a sheep, and a horse.

The second electrode may be made from gold.

The first electrode module may be provided with a component configured to generate or change the concentration of the chemical or biological target. In particular, the first electrode module comprising the component may be configured to generate a different concentration of the chemical or biological target from the concentration of the chemical or biological target on the second electrode.

The first electrode module may comprise a single first electrode, which may be made from silver. The first electrode may be coated in silver chloride.

Alternatively, the first electrode module may comprise two electrodes, one connected to the electrometer and one connected to the electronic component.

According to another aspect of the invention, there is provided a method for detecting the concentration of a chemical or biological target within a sample, the method comprising the steps of:

providing an electrochemical cell with a first electrode module and a second electrode;

providing an electronic component between the first electrode module and the second electrode;

introducing the sample into the electrochemical cell; measuring the potential difference between the first electrode module and second electrode;

converting the measured potential difference into a concentration of the chemical or biological target.

The method may further comprise the step of varying the resistance of the electronic component. The degree to which the resistor modifies the potential difference can be tuned by varying its resistance. In some embodiments, the resistance can be at zero, the electrochemical cell may be short circuited so the measured potential can become zero. As the resistance approaches infinity, the potential difference may tend to the OCP value. The magnitude of the resistor may be varied during measurements in order to optimise performance.

The method may further comprise the step of functionalising the second electrode with a biological recognition element such as an antibody. For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

The chemical or biological target may be a redox poising species.

The method may further comprise the step of providing a component configured to bind to the first electrode module to generate or change the concentration of the chemical or biological target on the first electrode module.

For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

Furthermore, according to the present invention there is provided a device for detecting the concentration of a chemical or biological target within a sample, the device comprising:

an electrochemical cell comprising a first electrode module and a second electrode both immersed in an electrolyte solution;

an electronic component provided between the first electrode module and the second electrode;

an electrometer configured to measure the potential difference between the first electrode module and the second electrode;

a memory configured to store data to provide conversion between measured potential difference and number of moles of chemical or biological target; and

a processor configured to identify the relevant data corresponding to the measured potential difference to process the concentration of the chemical or biological target within the sample.

The electronic component may be a variable resistor including either a rheostat or varistor or a switched group of resistors. Alternatively, the electronic component could also be a diode or a transistor.

The memory may further comprise data for different values of the resistance of the electronic component. The data storage may be in any suitable form of memory, either volatile or non-volatile, including but not limited to RAM, SRAM, ROM, EEPROM or Flash memory. The conversion data stored within the data storage comprises a set of calibration values correlating the potential difference measurements when known number of moles of a given electroactive species have been formed or depleted. This data may be retained in the form of individual data points or it may be processed to provide a functional description of the inter-relation between the potential difference and the number of moles of the electroactive species that are formed or depleted. The conversion data may be provided as a closed set of data with which all future potential difference measurements are compared. Alternatively, the data set may be augmented or replaced by periodic calibration of the device.

The second electrode may functionalised with a biological recognition element, which may be an antibody. Antibodies, including antibody fragments, suitable for use in the methods described herein are known in the art. For example, the antibody or the antibody fragment may be selected from one or more of the classes IgA, IgD, IgE, IgG and IgM. In some embodiments, the antibody or antibody fragment is of the IgG type. The antibody binds selectively to a target species or target analyte. The antibody or antibody fragment may be derived from a mammal, including, but not limited to, a mammal selected from a human, a mouse, a rat, a rabbit, a goat, a sheep, and a horse.

The second electrode may be made from gold.

The first electrode module may be provided with a component configured to generate or change the concentration of the chemical or biological target. In particular, the first electrode module comprising the component may be configured to generate a different concentration of the chemical or biological target from the concentration of the chemical or biological target on the second electrode.

The first electrode module may comprise a single first electrode, which may be made from silver. The first electrode may be coated in silver chloride.

Alternatively, the first electrode module may comprise two electrodes, one connected to the electrometer and one connected to the electronic component.

According to the present invention there is provided a method for detecting the rate of production or depletion of a chemical or biological target within a sample, the method comprising the steps of:

providing an electrochemical cell with a first electrode module and a second electrode;

providing an electronic component between the first electrode module and the second electrode;

introducing the sample into the electrochemical cell;

repeatedly measuring the potential difference between the first electrode module and second electrode;

processing the potential difference measurements to identify a rate of change of potential difference and converting that rate of change of potential difference into a rate of production or depletion of the chemical or biological target.

The method may further comprise the step of varying the resistance of the electronic component. The degree to which the resistor modifies the potential difference can be tuned by varying its resistance. In some embodiments, the resistance can be at zero, the electrochemical cell may be short circuited so the measured potential can become zero. As the resistance approaches infinity, the potential difference may tend to the OCP value. The magnitude of the resistor may be varied during measurements in order to optimise performance.

The method may further comprise the step of functionalising the second electrode with a biological recognition element such as an antibody. For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

The chemical or biological target may be a redox poising species.

The method may further comprise the step of providing a component configured to bind to the first electrode module to generate or change the concentration of the chemical or biological target on the first electrode module.

For use in immuno-sensing, the second electrode can be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich. For example, the secondary recognition element may bind to a target analyte bound to the biological recognition element. In some embodiments, the bound secondary recognition element may comprise the component. The secondary recognition element may be an antibody or a fragment thereof. The target analyte may be any analyte that can be detected using an antibody or antibody fragment.

Additionally or alternatively, the component may bind directly to the second electrode or it may bind to the biological recognition element. The component is capable of generating an electroactive species. This may take place in a second step where the sensor may be incubated in a different solution. The generated electroactive species may be solution or surface bound, and it may be able to change the redox poise of the second electrode.

Furthermore, according to another aspect of the present invention, there is provided a device for detecting the rate of production or depletion of a chemical or biological target within a sample, the device comprising:

an electrochemical cell comprising a first electrode module and a second electrode both immersed in an electrolyte solution;

an electronic component provided between the first electrode module and the second electrode;

an electrometer configured to measure the potential difference between the first electrode module and the second electrode;

a memory configured to store data to provide conversion between measured rate of change of potential difference and rate of production or depletion of the chemical or biological target; and

a processor configured to identify the relevant data corresponding to the measured rate of chance of potential difference to process the rate of production or depletion of the chemical or biological target within the sample.

The electronic component may be a variable resistor including either a rheostat or varistor or a switched group of resistors. Alternatively, the electronic component could also be a diode or a transistor.

The memory may further comprise data for different values of the resistance of the electronic component. The data storage may be in any suitable form of memory, either volatile or non-volatile, including but not limited to RAM, SRAM, ROM, EEPROM or Flash memory. The conversion data stored within the data storage comprises a set of calibration values correlating the potential difference measurements when known number of moles of a given electroactive species have been formed or depleted. This data may be retained in the form of individual data points or it may be processed to provide a functional description of the inter-relation between the potential difference and the number of moles of the electroactive species that are formed or depleted. The conversion data may be provided as a closed set of data with which all future potential difference measurements are compared. Alternatively, the data set may be augmented or replaced by periodic calibration of the device.

The second electrode may functionalised with a biological recognition element, which may be an antibody. Antibodies, including antibody fragments, suitable for use in the methods described herein are known in the art. For example, the antibody or the antibody fragment may be selected from one or more of the classes IgA, IgD, IgE, IgG and IgM. In some embodiments, the antibody or antibody fragment is of the IgG type. The antibody binds selectively to a target species or target analyte. The antibody or antibody fragment may be derived from a mammal, including, but not limited to, a mammal selected from a human, a mouse, a rat, a rabbit, a goat, a sheep, and a horse.

The second electrode may be made from gold.

The first electrode module may be provided with a component configured to generate or change the concentration of the chemical or biological target. In particular, the first electrode module comprising the component may be configured to generate a different concentration of the chemical or biological target from the concentration of the chemical or biological target on the second electrode.

The first electrode module may comprise a single first electrode, which may be made from silver. The first electrode may be coated in silver chloride.

Alternatively, the first electrode module may comprise two electrodes, one connected to the electrometer and one connected to the electronic component.

### BRIEF DESCRIPTION OF THE FIGURES

The invention will now be further and more particularly described, by way of example only, and with reference to the accompanying drawings, in which:

FIG. 1 provides an illustration of the sensing apparatus according to the present invention;

FIG. 2 provides an alternative embodiment of the sensing apparatus according to FIG. 1;

FIG. 3 shows experimental data of the measured potential of a working electrode functionalised with a biological recognition elements spiked with various concentrations of C-reactive protein (CRP);

FIG. 4 shows a calibration curve for potentials taken at 12 seconds for different concentrations of CRP;

FIG. 5 shows a liquid handling cartridge according to an aspect of the present invention;

FIG. 6A shows a positive control: Open Circuit Potential vs Ag|AgCl measured for a platypus chip functionalised with a-CRP-polyHRP first in PBS and then in TMB blotting solution;

FIG. 6B shows a positive control: Open Circuit Potential vs Ag|AgCl measured for a platypus chip functionalised with Stabilblock first in PBS and then in TMB blotting solution;

FIG. 7 shows a plot of OCP response for PBS vs 1 mM Fe(CN)₆^{3-/4-} for difference resistances;

FIG. 8 shows a plurality of OCP traces on 2 mm Au2 chip in 1 mM Fe(CN)₆^{3-/4-} coupled to Ag|AgCl reference with varying resistances;

FIG. 9 shows a plurality of OCP traces on 2 mm Au2 chip in PBS coupled to Ag|AgCl reference with varying resistances;

FIG. 10 provides a graph illustrating OCP (redox)—OCP (PBS) as a function of resistance;

FIG. 11 shows a plurality of OCP traces for sensors that have been incubated in different concentrations of CRP before and after addition of TMB Blotting solution;

FIG. 12 shows a calibration curve of OCP steady state for different concentrations of CRP;

FIG. 13 provides data showing a comparison between two electrodes to three electrode setups in 1 mM Ferri/Ferro in PBS for a 2 mm Au macro;

FIG. 14 shows a calibration curve for assay on array using OCP two electrode setup;

FIG. 15 shows a plurality of OCP traces for assay on array using two electrode setup;

FIG. 16 provides a plot of internal pseudo-reference potential in TMB Blotting solution as function of [KCl];

FIG. 17 shows an assay performed on arrays/TMB for CRP;

FIG. 18 shows a plurality of OCP traces for CRP assay;

FIG. 19A provides an embodiment that shows an configuration of multiple electrodes; and

FIG. 19B provides an alternative embodiment that shows an alternative configuration of multiple electrodes.

### DETAILED DESCRIPTION OF THE FIGURES

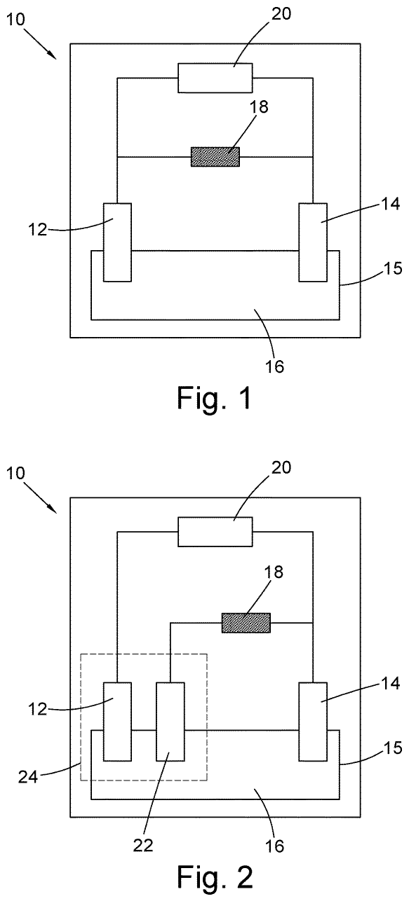

Referring to FIG. 1, there is provided an apparatus 10 and method for sensing the formation or depletion of a chemical or biological target. The potentiometric apparatus 10 of the present invention comprises: a first electrode module, which in this embodiment is implemented solely by a first electrode 12; a second electrode 14 and a container 15. The container 15 contains an electrolyte solution 16. The container 15 may be a water bath such as a thermostat water bath. The water bath may be configured to regulate the temperature of the first electrode 12 and the second electrode 14. The first electrode 12 and the second electrode 14 are immersed in an electrolyte solution 16 such that a current can pass between the first electrode 12 and the second electrode 14.

By immersing the electrodes in an electrolyte solution, a potential difference is set up which generates a flow of current between the first and second electrodes through the variable resistor 18. The potential difference that exists between the first and second electrodes depends on the materials of the electrodes and the solution that the electrodes are placed in.

A variable resistor 18, such as a 5 M Ohm resistor, is provided between the first electrode 12 and the second electrode 14. Placing a resistor 18 between the first 12 and second 14 electrodes modifies the potential difference between the first 12 and second 14 electrodes away from its equilibrium (the OCP value) as it allows current to pass in a closed circuit. The current flowing can be determined by the value of the resistor and the potential difference between the electrodes according to Ohm's Law.

A species can be provided which is configured to bind to the second electrode 14 to generate or change the concentration of an electroactive target in solution and/or on the second electrode 14. It is capable of changing the potential at the second electrode 14 relative to the first electrode 12. As illustrated in FIGS. 1 and 2, the second electrode 14 is a working electrode.

Electroactive species such as an electroactive precipitate, may be capable of changing the potential of the working electrode via electron transfer. Examples of an electroactive species may be ferrocyanide, ferricyanide, ferrocene, ferrocenium, transition metal complexes, oxidised and reduced forms of DAB, AEC or mixtures or precipitates containing one or more redox species.

As illustrated in FIGS. 1 and 2, electrode 12 is a reference electrode. This electrode 12 is either a stand-alone electrode, as indicated in the embodiment of FIG. 1 or it may form part of an electrode module 24 as illustrated in the embodiment of FIG. 2. In each of these embodiments, the first electrode 12 can be made from any material that is capable of maintaining a constant and predictable potential in an electrolyte solution. In addition, the reference electrode may have a standard reduction potential that can be different to that of the generated electroactive species.

The potential at each electrode is determined by all redox half-cell reactions occurring at the electrode/solution interface. These half-cell reactions comprise species interchanging electrons with the electrode surface. The kinetics of each half-cell reaction depends on the accessible redox states, activity and diffusion coefficient of each species as well as the catalytic properties of the electrode material, which can strongly affect the mechanism by which the redox reaction occurs. The potential difference reaches an equilibrium value when the net current of all reactions at each electrode matches the current drawn through the variable resistor. As the component that is bound to the second electrode generates or depletes one or more redox species, the potential of the system shifts so as to return to equilibrium. The magnitude of this change is proportional to the change in the concentration of the chemical or biological target.

As shown in FIG. 1, a measuring device 20 is provided between the first electrode 12 and the second electrode 14. The measuring device 20 is an electrometer such as the OCP mode of a PalmSense4™ device. The measuring device may be a USB and/or a battery powered potentiostat or a galvanostat and optionally, a Frequency Response Analyser (FRA) may be provided within the device for electrochemical measurements. The electrometer is able to resolve potentials accurately to at least 1 mV and preferably at 0.1 mV.

The measuring device such as the PalmSens4™ may have a large potential range for example, the potential range may be between −5V to 5V or it may be between −10V to 10V. The current range of the measuring device 20 may be between 100 pA to 10 mA with a high resolution and low noise.

The measuring device 20 is configured to measure the potential difference as a function of time across the resistor 18 before and during the formation or depletion of an electroactive species, where a change in the potential difference equal to or above a threshold level indicates the formation or depletion of the electroactive species. The threshold value can be between 1 mV and 500 mV. The potential vs time trace can be processed algorithmically to recover information about the kinetic process of species formation or depletion. This can be related to the amount of target that is present in the sample.

Referring to FIG. 2, there is provided a potentiometric apparatus 10 and method for sensing the formation or depletion of chemical or biological target according to the present invention. The potentiometric apparatus 10 comprises: a first electrode module 24 including a first electrode 12 and a further electrode 22; a second electrode 14 and a container 15. The container 15 such as a water bath can contain an electrolyte solution 16. The first electrode 12, the second electrode 14 and the further electrode 22 are immersed in an electrolyte solution such that a current can pass between the further electrode 22 and the second electrode 14.

The further electrode 22 may be a reference electrode of the same or a different material to the first electrode 12. Alternatively, the further electrode 22 may be an electrode that undergoes a change in potential due to the formation of the electroactive species, for example an electroactive precipitate.

In addition, a measuring device 20 is provided between the first electrode 12 and the second electrode 14, as shown in FIG. 2. The measuring device 20 is configured to measure the potential difference as a function of time across the resistor 18 before and during the formation or depletion of an electroactive species.

Other circuit components (not shown in the accompanied drawings) may be provided to the potentiometric apparatus 10 either alone or in combination to couple the working electrode 14 and the reference electrode 12 together. These may include capacitors and/or diodes.

The apparatus according to FIGS. 1 and 2 may be used for immuno-sensing assays. The second electrode 14 is functionalised with a biological recognition element such as a primary antibody. The primary antibody is immobilised onto the surface of the working electrode 14 via a biological or chemical linker. In some examples, the primary antibody may be immobilised on the working electrode surface by EDC/NHS activation, other linking chemistries (maleimide, click chemistry, epoxy, tosyl, chloromethyl, iodoacetamide), biotin-streptavidin or by passive adsorption.

The working electrode may be made of an inert material such as gold and the working electrode can be functionalised with a biological recognition element. For example, the biological recognition element may be a protein, a peptide, an antibody or a fragment thereof or a nucleic acid.

A sandwich-format immunoassay may be performed with secondary recognition elements closing the sandwich i.e. the secondary recognition element binding to the biological recognition element. In some embodiments, the bound secondary recognition element may comprise the component. The secondary recognition element may be a secondary antibody or a fragment thereof. The component such as an enzyme may be bound to the secondary antibody.

The method may be carried out such that the target analyte, the secondary antibody and enzyme label are incubated in a single solution for a period of time before contacting the primary antibody which has been immobilised on the surface of the working electrode.

The enzyme may be selected from but is not limited to horseradish peroxidase (HRP), alkaline phosphatase, glucose oxidase and β-galactosidase; enzyme label may be in a polymeric form. Any suitable substrate may be used, for example DAB (3,3′-diaminobenzidine), stabilised DAB, AEC (3-amino-9-ethylcarbazole) or BCIP (5-bromo-4-chloro-3-indolyl phosphate), NBT (nitro-blue tetrazolium chloride), TMB (3,3′,5,5′-tetramethylbenzidine), ELF (enzyme-labelled fluorescence) or OPD (o-phenylenediamine dihydrochloride). The substrate may be stabilised DAB, for example ImmPact™ DAB. The enzyme substrate may be TMB (tetramethylbenzidine), which can be used as a substrate without requiring a further redox probe and can therefore be directly measured in a buffered solution.

Other enzymes that are routinely used to label antibodies are known in the art, including alkaline phosphatase, glucose oxidase and β-galactosidase, and may be used in the methods described herein when combined with an appropriate substrate, for example, one that forms an insoluble precipitate in the presence of the enzyme.

An example of a non-electroactive species may be the target analyte-secondary antibody-enzyme complex which is capable of binding to primary antibody immobilised on the surface of the working electrode.

In some embodiments, a binding event between an immobilised primary antibody on the surface of the second electrode such as the working electrode with a target and a secondary antibody-enzyme complex may be detected using an electrochemical measurement technique.

The method may comprise contacting a primary antibody immobilised on the surface of the working electrode with the target analyte, the secondary antibody and the enzyme label to create a sandwich complex bound to the electrode surface. The sandwich complex formed on the surface of the electrode can contact with a substrate for the enzyme label where the substrate is converted by the enzyme into an insoluble precipitate on the electrode surface. This can significantly amplify the signal which can be detected by various means including electrochemical detection methods such as EIS, voltammetry e.g. differential pulse voltammetry (DPV), square wave voltammetry (SWV), cyclic voltammetry (CV), chronoamperometry, chronopotentiometry, Open Circuit Potential (OCP).

In some embodiments, the target, secondary antibody and an enzyme label may be incubated in a single solution before contacting with the primary antibody immobilised on the surface of the electrode.

The single incubation step has significant advantages over the traditional multi-step process. Firstly, by adding all the reagents together (antigen, secondary antibody, enzyme) there are less steps required in order to perform the sandwich immunoassay. This can save time from the incubation, reduce the washing steps and therefore improve reproducibility and reduce variation. In addition, for PoC (point of care) devices where reagent space is limited, by having all the components together can save precious space in a cartridge system and significantly simplifies workflow. This in turn, leads to reduced error from the system and further improving the reproducibility of the assay. Advantageously, the methods provided herein do not require electropolymerisation of the insoluble precipitate.

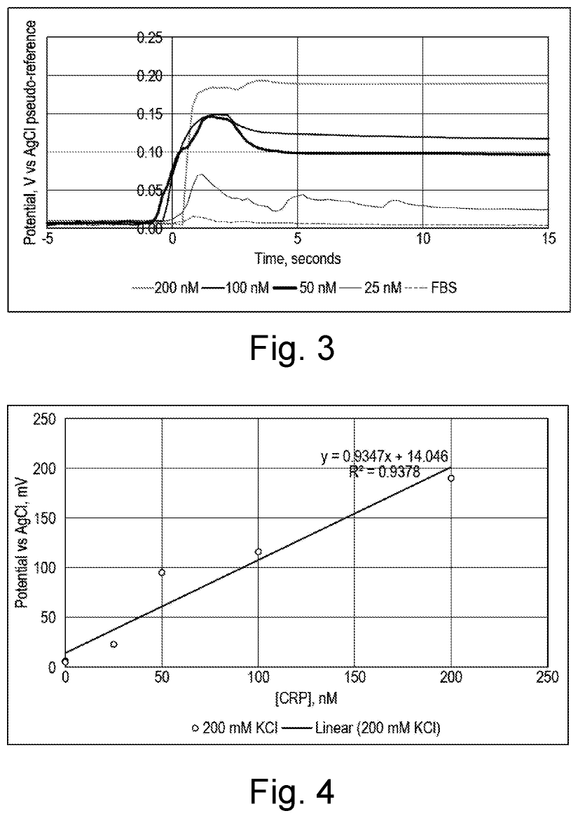

Referring to FIG. 3, there is provided experimental data of the measured potential of a working electrode functionalised with a biological recognition elements spiked with various concentrations of an analyte. By way of example only, the target analyte can be a C-reactive protein (CRP). The target analyte can be present in a sample obtained as whole blood, plasma or serum. The target analyte may also be troponin. The potential vs time trace as shown in FIG. 3 can be processed algorithmically to recover information about the kinetic properties of the electroactive species formation or depletion at the interface of the working electrode.

Referring to FIG. 3, the graph provide traces in buffered saline solution before and after injection of the precursor of the electroactive species at Time=0 for different concentrations of target. Referring to FIG. 3, arrays of gold working electrodes functionalised with primary antibodies are incubated in solutions containing various concentrations of the target. Also present in the incubation is a secondary antibody specific to the target that has been functionalised with a redox enzyme. After 10 minutes of incubation, the arrays can be washed to remove non-specific interaction and the measurement apparatus can be set up as illustrated in FIGS. 1 and 2.

As shown in FIG. 3, the steady state potential can be measured at different concentrations of the target during the formation or depletion of the electroactive species on the surface of the electrode. As used herein and unless otherwise specified, the term “steady state” may be referred to when equilibrium is reached between the rate of formation of the electroactive species and the rate of depletion of the electroactive species. The steady state potential that is reached can depend on a combination of at least two factors. The first is the rate of formation of the electroactive species e.g. the electroactive precipitate, which is controlled by the number of moles per surface area of the species capable of generating electroactive precipitates on the electrode. This may also mean the number of electrons transferring to the surface of the electrode or the number of charged molecules at the surface of the electrode or the number of moles of electroactive precipitates per surface area/electrode density.

The second factor is the rate of depletion of the precipitate, which is controlled by the current drawn through the variable resistor which oxidises or reduces the precipitate back to a neutral non-electroactive form. The current drawn is dependent on the magnitude of the resistance chosen. By fixing the magnitude of the variable resistor to, for example, 5 M ohms, the steady state is now only a function of the first factor and thus quantitatively measures the concentration of the antigen in solution.

The potential across the 5 M Ohm resistor is measured before and after injection of the solution which happened at Time=0, as shown in FIG. 3. As shown in FIG. 3, the potential changed immediately after injection due to the immobilised enzyme producing an electroactive precipitate that has known redox poise.

As an alternative to measuring a steady state potential, quantitative bio-sensing can be achieved by computing the rate of change in the potential during the time that it is changing. For example, after the electroactive species has started to form as an electroactive precipitate and the potential has changed, but before a steady-state has been reached. This can manifest as a simple linear fit to the initial rate of change, or could use a more complex functional form to fit the trace such as polynomial, exponential etc.

Referring to FIG. 4, a standard calibration curve is provided for an assay of C-Reactive protein in the 0 nM to 100 nM range. A 5 M Ohm resistor is selected to couple the working electrode to the reference electrode. In some instances, the 5 M Ohm resistor is used because it is optimum for this assay. The calibration curve as shown in FIG. 4 has been constructed by taking the potential difference at Time=12 seconds at different concentrations e.g. 25 nM, 50 nM, 100 nM and 200 nM of CRP spiked into FBS.

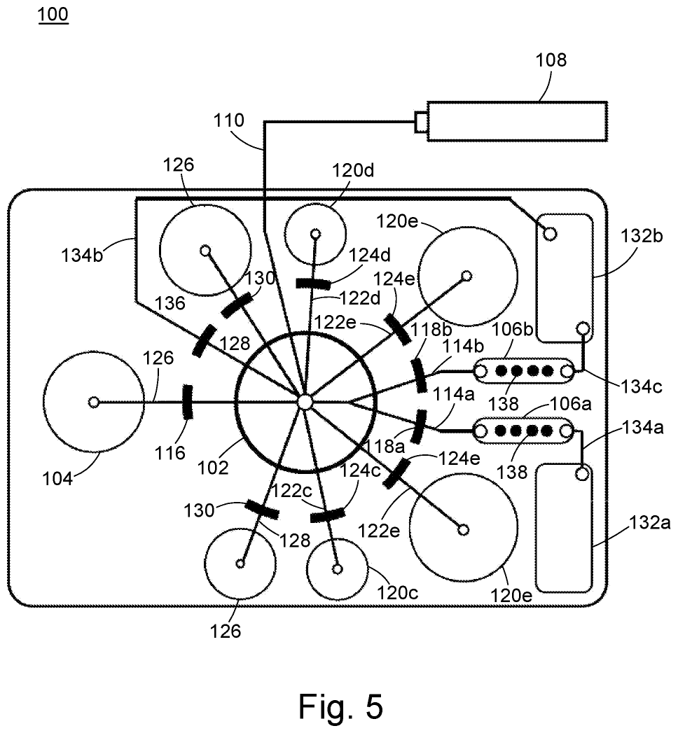

Referring to FIG. 5, there is provided a liquid handling cartridge 100. The liquid handling cartridge 100 comprises a main chamber 102; a sample chamber 104 for receiving a sample; one or more measurement chambers 106a/106b comprising the apparatus 10 and/or device as described in FIGS. 1 to 4 for performing measurements on the sample; a variable pressure source conduit 110 connecting the main chamber 102 to a variable pressure source 108; a sample chamber conduit 112 which fluidically connects the sample chamber 104 to the main chamber 102; a sample chamber conduit valve 116 for opening and closing the sample chamber conduit 112; a respective measurement chamber conduit 114a/114b for each measurement chamber 106a/106b, wherein each respective measurement chamber conduit 114a/114b fluidically connects the respective measurement chamber 106a/106b to the main chamber 102; and a respective measurement chamber conduit valve 118a/118b for opening and closing each respective measurement chamber conduit 114a/114b. The variable pressure source conduit 110 is connected to the main chamber 102 at the top of the main chamber.

The main chamber 102 is arranged to receive a fluid from the sample chamber 104 when the sample chamber conduit valve 116 is open and a negative pressure change is applied to the main chamber 102 via the variable pressure source conduit 110. Further, the one or more measurement chambers 106a/106b are arranged to receive the fluid from the main chamber 102 when the respective measurement chamber conduit valves 118a/118b are open and a positive pressure change is applied to the main chamber 102 via the variable pressure source conduit 110.

The liquid handling cartridge 100 further comprising one or more reagent chambers 120a/120b; a respective reagent chamber conduit 122a/122b for each reagent chamber 120a/120b, wherein the respective reagent chamber conduit 122a/122b fluidically connects the respective reagent chamber 120a/120b to the main chamber 102; and a respective reagent chamber conduit valve 124a/124b for opening and closing each respective reagent chamber conduit 122a/122b.

The liquid handling cartridge 100 comprises two measurement chambers 106a/106b. The two measurement chambers 106a/106b are known as a first measurement chamber 106a for performing a first measurement on the sample and a second measurement chamber 106b for performing a second measurement on the sample.

The liquid handling cartridge 100 further comprises a first respective measurement chamber conduit 114a which fluidically connects the first measurement chamber 106a to the main chamber 102; a second respective measurement chamber conduit 114b which fluidically connects the second measurement chamber 106b to the main chamber 102; a first respective measurement chamber conduit valve 118a for opening and closing the first respective measurement chamber conduit 114a; and a second respective measurement chamber conduit valve 118b for opening and closing the second respective measurement chamber conduit 114b.

As shown in FIG. 5, the liquid handling cartridge 100 further comprising a first dedicated reagent chamber 120c for a reagent to be used only in a diagnostic test to be performed in a first measurement chamber 106a; a second dedicated reagent chamber 120d for a reagent to be used only in a diagnostic test to be performed in the second measurement chamber 106b; two shared reagent chambers 120e for reagents to be used in the diagnostic tests to be measured in both the first and second measurement chambers 106a/106b; and two mixing chambers 126. Conduits 122c/122d/122e/128 and conduit valves 124c/124d/124e/130 for the corresponding chambers are provided.

The liquid handling cartridge 100 further comprises a first waste chamber 132a and a second waste chamber 132b. A first waste chamber conduit 134a fluidically connects the first waste chamber 132a to the first measurement chamber 106a. A second waste chamber conduit 134b fluidically connects the second waste chamber 132b to the main chamber 102 and a third waste chamber conduit 134c fluidically connects the second waste chamber 132b to the second measurement chamber 106b. A second waste chamber conduit valve 136 is configured to open and close the second waste chamber conduit 134b.

The variable pressure source conduit 110 is connected to a variable pressure source 108. The conduit valves are pinch valves and are configured in a circular array. Each measurement chamber 106a/106b comprises a plurality of electrodes 138. The chambers of the liquid handling cartridge 100 comprise gas exchange holes for allowing air or any other ambient gas to enter and exit each chamber to balance a pressure change resulting from liquid (such as sample or reagent) entering the respective chambers. The gas exchange holes are not essential, while other gas exchange means such as air vents can be used instead or in addition to holes.

### EXAMPLES

### Example 1. Experimental Data on 3 Electrode System

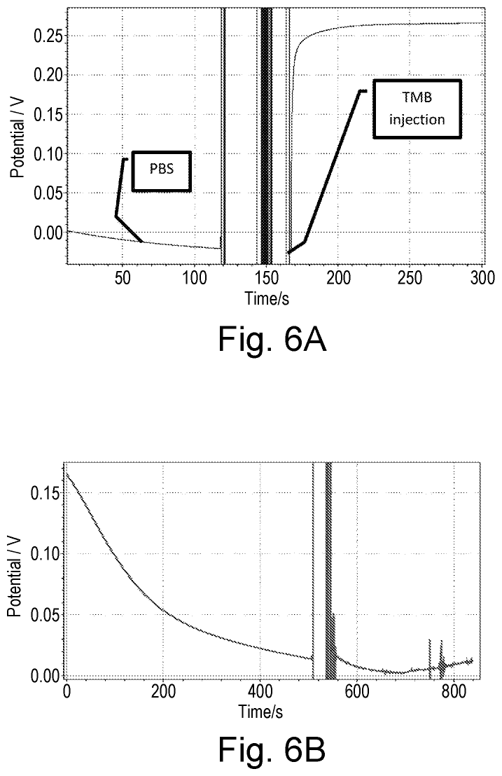

Electrodes that comprises captured HRP-tagged secondary antibody on their surface can show a change in open circuit potential (OCP) upon incubation in Pierce Ultra TMB blotting solution, which contains TMB, H₂O₂ and various proprietary sugars, polymers and detergents which stabilise the precipitate. FIG. 6A shows the OCP response for a platypus gold SPR chip that has been incubated in 1:1000 a-CRP-polyHRP for 30 m, first measured in PBS and then on incubation in 100% TMB blotting solution. A sharp rise from the initial value of around −50 mV is observed, with a plateau at around 260 mV. No change in the response can be observed for negative control in which the sensor was functionalised only with Stabilblock (FIG. 6B). The OCP changes due to the accumulation of a positively charged charge transfer complex ([TMB,TMB(II)]) which is a precipitate that exhibits 2 electron reversible redox process with E(½)=310 mV vs Ag|AgCl.

The method for sensing as disclosed herein could be advantageous because it simplifies the workflow [incubation, wash, TMB]; takes less time by parallelising measurement with precipitate formation (roughly 3 minutes shorter in current workflow); removes the need to accurately control the precipitation time, improving accuracy. In addition, the method as disclosed herein does not suffer from loss of precipitate due to flaking from fluidics or dissolution in measurement buffer. This could be done on a microelectrode without as much signal loss as amperometric methods.

Furthermore, the method of the present invention can be performed in a two electrode configuration with a high input impedance electrometer rather than potentiostats, making the cost of reader cheaper. It may also be much easier to measure electrodes in parallel, using sampling between electrodes or poly-electrometer.

In addition, the method and device as described herein is non-peturbative so it can be combined with other measurements to improve reliability, extend the linear range, or give faster results in clear cases. The parameters could be extracted from the rising part of the OCP trace to give additional kinetic information that is not readily available in other methods.

The OCP is a mixed potential which is determined by many half reactions at the electrode-solution interface. On a platinum UME, it can be shown that increasing oxygen concentration caused a positive shift in OCP and can be concluded that the dominant anodic half reaction can be water oxidation, with the OCP showing an expected shift of −59 mV/pH. As these half reactions are inner sphere, the state of the surface influences the relative size of their currents and thus the equilibrium potential where I_{anodic}=−I_{cathodic}.

The weakness of the half reactions on relatively inert materials such as Au and Pt means that equilibration is slow and baselines vary. Moreover, the strength of the baseline OCP poise may be variable depending on pH, [O₂] and other species, which means that OCP will respond at variable speeds to accumulation of precipitate and may reach a different final steady state. A ‘sensitivity factor’=ΔE_{OCP}/Δᵢₘᵢₓ can be used to quantify this property.

Adding stronger half-reactions at the interface can set up a more robust, stable baseline value for OCP. This comes at the cost of sensitivity as a greater amount of precipitate can be required to poise the signal potential. A possible constraint is that the baseline potential should be significantly different to the signal potential. The obvious additive to effect a redox poise would be Fe(CN)^{3-/4-} for which E_{1/2}=210 mV. However, this value can be quite similar to that of TMB (˜300 mV at pH 5.5). Moreover, the components of the TMB blotting solution can be finely tuned to maximise the production of a charge transfer complex and stabilise peroxide.

### Coupling the Sensor to Another Reference Electrode

One way to control the initial redox poise at the electrode without adding species is to mix in the redox potential of another system to the measured OCP. A very basic system was designed to test this, which included a second reference electrode coupled to the working electrode by a variable resistor. The reference electrode has a known, stable redox poise and the variable resistor can be used to tune its contribution to get reasonable sensitivity. In PBS, the weak half-reactions which are kinetically slow will contribute negligibly with respect to the strong Nernst equation at the reference, but on addition of TMB precipitate, the half-reactions at the working electrode will provide a significant contribution to the OCP signal due to their faster kinetics.

### Demonstration with Solution Phase Redox Probe

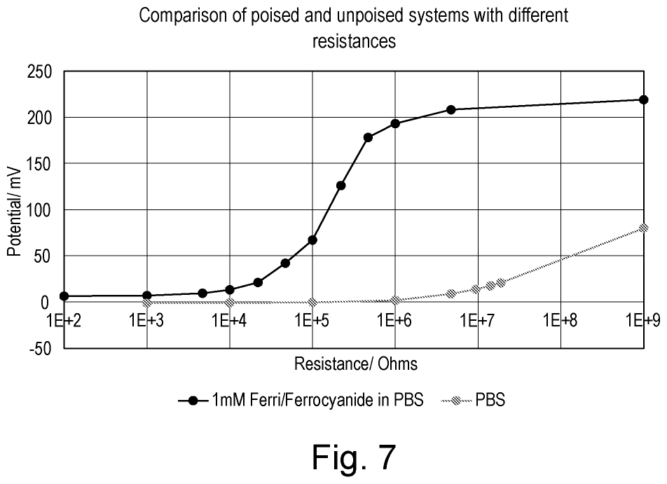

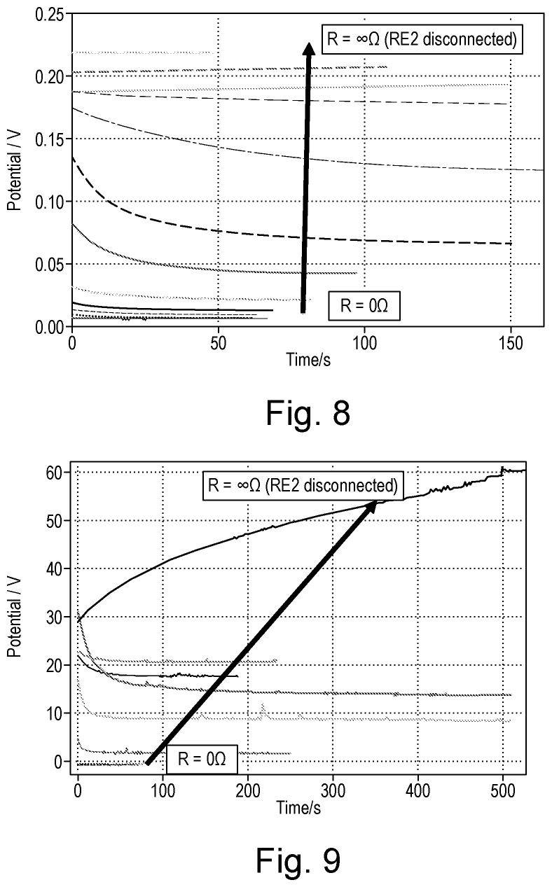

The value of OCP at a 2 mm Au2 chip cleaned in O2 plasma was measured in both 10×PBS and in 1 mM Fe(CN)^{3-/4-6} in a PBS supporting electrolyte, using the three electrode system setup as shown in FIG. 2 with different values for the varying resistor. The results are plotted in FIG. 7, using the final OCP values after the signal had stabilised. The raw OCP traces are shown in FIGS. 8 and 9. As expected, the mixing of a second reference leads to much faster equilibration of the OCP in PBS. The robustness of the OCP as signal is evident: it was not possible to reach a steady value with infinite resistance even after 15 minutes in PBS, whereas all traces stabilised in less than a minute when RE2 was included. In the traces measured with the redox couple, the steady state is reached most slowly for intermediate resistances.

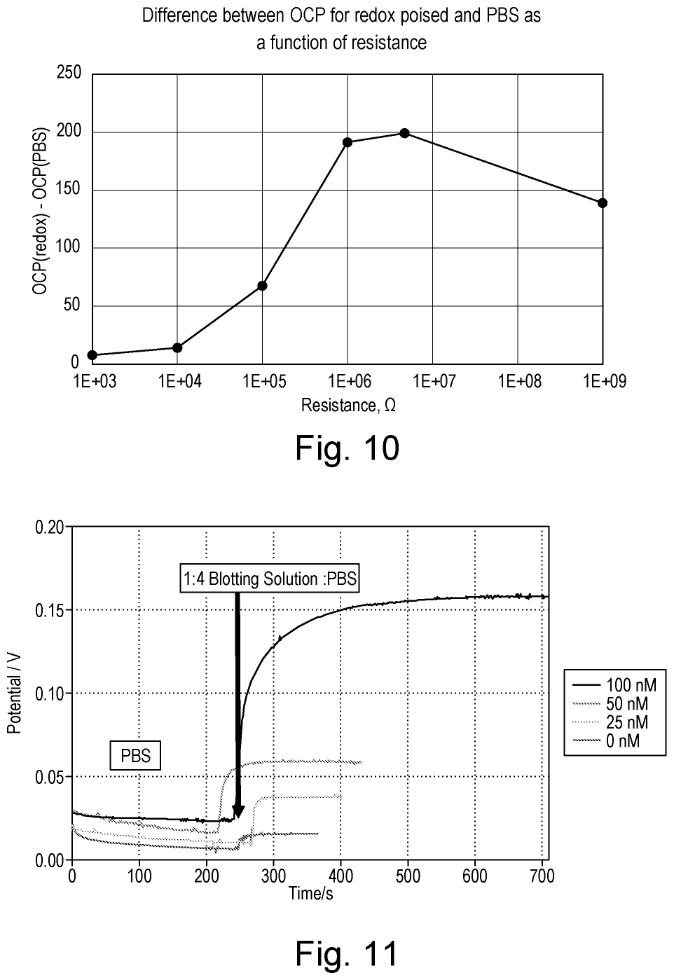

As shown in FIG. 10, there is shown that resistances can be chosen that favour fast kinetics of poised potential over slow mixed potentials and removes baseline drift. FIG. 10 shows the difference signal—baseline as a function of resistance for the Ferri/Ferrocyanide system.

### Assay

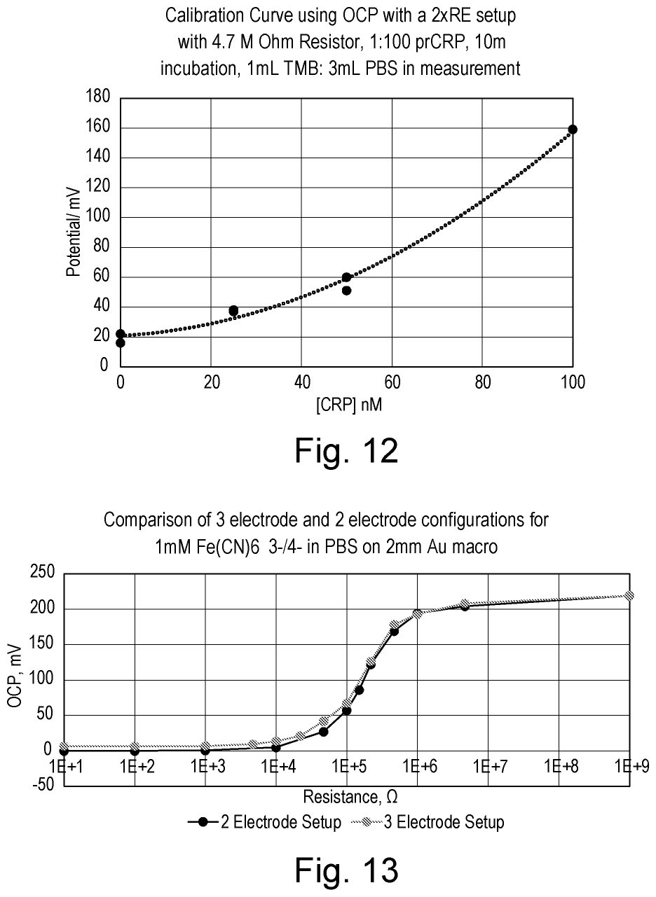

10 mm×10 mm Au2 squares were cleaned in O2 plasma before being incubated in 1:100 pr-CRP antibody in carbonate buffer for 1 hr. They were washed in PBST and then blocked with Stabilblock for 30 m. The sensors were then incubated for 10 minutes in different samples that contained 0, 25, 50 or 100 nM CRP at 1:500 dilution in PBST with 1:2000 sec-CRP ab and 1:3000 poly-HRP. They were washed in PBST for 5 minutes and left in PBS until measurement, which was done immediately. For measurement, the squares were placed in an incubation cell with an O-ring that limited the diameter to 2 mm with external Ag|AgCl 3M KCl references. A resistor of 4.7 MS2 was chosen. The OCP was measured first in PBS and then after around 200 seconds, a small amount of TMB blotting solution was injected and vigorously mixed to give a final ratio of 1:4 TMB:PBS. Exemplary OCP traces for each concentration are shown in FIG. 11, and a calibration curve is plotted in FIG. 12. This preliminary data shows that OCP is sensitive enough to quantify small changes in the concentration of an analyte via monitoring the redox poise of the interface due to precipitation of TMB.

In conclusion, OCP can be used to measure the concentration of CRP, which permits a much simpler workflow (incubation, washing, precipitation) and does not require a potentiostat. As OCP is poorly defined for non-poised systems, a method was developed that avoided the addition of any species into solution and used a second reference electrode, which provided the necessary robustness for use of OCP as a transduction signal.

### Example 2. Two Electrode System

The three electrode system that was discussed in example 1 is equivalent to the two electrode system shown in FIG. 1. The configuration as illustrated in FIG. 1 is much simpler as it only needs two electrodes in the cell, so can be incorporated and tested on the existing arrays. FIG. 13 shows plots of OCP against the value of the variable resistor for both configurations measuring 1 mM Fe(CN)^{3-/4-} in PBS, demonstrating their parity.

### Assay on Array in 2 Electrode Setup

The following method describes an example of assaying a sample in a two-electrode setup.
Cleaning

Grey dielectric 5 electrode 2-layer Au-2 arrays (WE radius=0.35 mm) were washed with DI water, dried with N2 and placed in O2 plasma (Cleaner 2) for 30 m, lowest power, pressure=0.3 mbar.
Assembly

The arrays were assembled with RE/CE layer and flow cell v4 in under 7 minutes.
Incubation

They were then incubated in 1:100 a-CRP primary (in carbonate buffer) for 1 hr, followed by washing in PBST. The sensors were then left in Stabilblock for >20 m.
Assay

The were then incubated for 10 m with 1:1000 plasma+1:1000 sec a-CRP, 1:3000 poly-HRP. CRP concentrations were 31, 62.5, 125, 250, 500 and 1000 nM. The arrays were washed in PBST and left in PBS for a short time before measurement.
Measurement

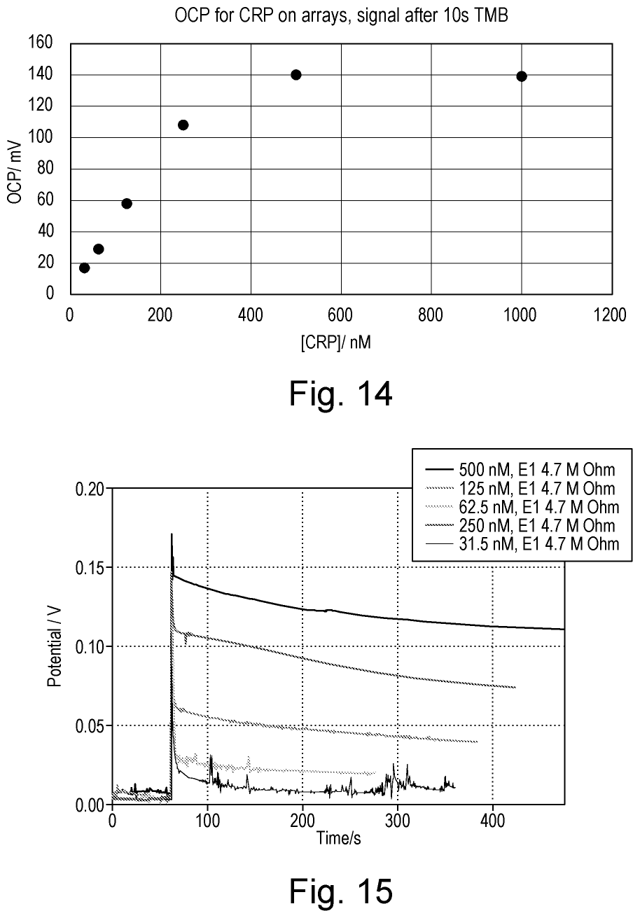

Measurement can be done in random order and comprised taking OCP before and after injection of pure TMB blotting solution until a stable signal was observed. The calibration curve is shown in FIG. 14 and the measured traces in FIG. 15. Internal RE was used.

### Reference Electrode Stabilisation

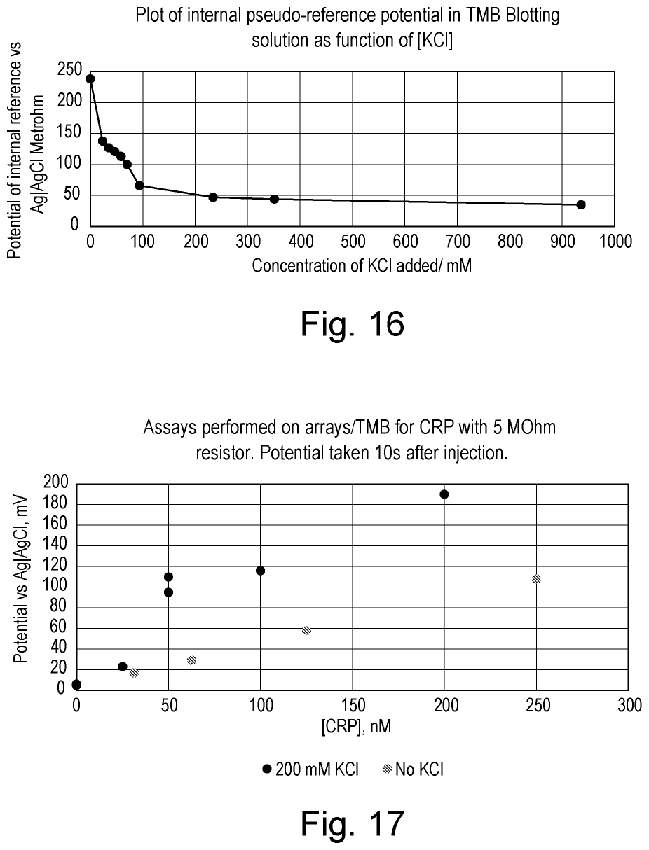

In some instances in both of the above assays, the OCP signals may not reach the expected potential for TMB vs AgCl (around 250 mV), and also that they may lose potential over time, even for highly saturated conditions. A possible reason for this was found to be due to the AgCl internal pseudo-reference not holding the expected potential in TMB Blotting solution. This may be because the solution does not contain chloride anions. FIG. 16 shows how the potential of the internal reference changes (vs Metrohm Ag|AgCl, 3M standard) for added concentrations of KCl to the TMB solution. It can be seen that a concentration of around >100 mM KCl is required for the reference to reach its expected value (50-60 mV). No immediate instability of the TMB solution was seen on addition of 200 mM chloride.

### Assay on Array in 2 Electrode Setup with Stabilised Reference

Cleaning

Blue dielectric 5 electrode 2-layer Au-2 arrays (WE radius=0.35 mm) were washed with DI water, dried with N2 and placed in O2 plasma (Cleaner 2) for 30 m, lowest power, pressure=0.3 mbar.
Assembly

The arrays can be assembled with reference electrode (RE)/counter electrode (CE) layer and flow cell v4 in under 5 minutes.
Incubation

The sample can be incubated in 1:100 a-CRP primary (in carbonate buffer) for 1 hr, followed by washing in PBST. The sensors were then left in Stabilblock for >20 m.
Assay

The sample can be incubated for 10 m with 1:1000 plasma+1:1000 sec a-CRP, 1:3000 poly-HRP. CRP concentrations were 0 nM+FBS, 25 nM, 50 nM, 100 nM and 200 nM. The arrays were washed in PBST and left in PBS for a short time before measurement.
Measurement

Measurement can be done in random order and comprised taking OCP before and after injection of TMB that had been spiked with 234 mM KCl until a stable signal was observed.

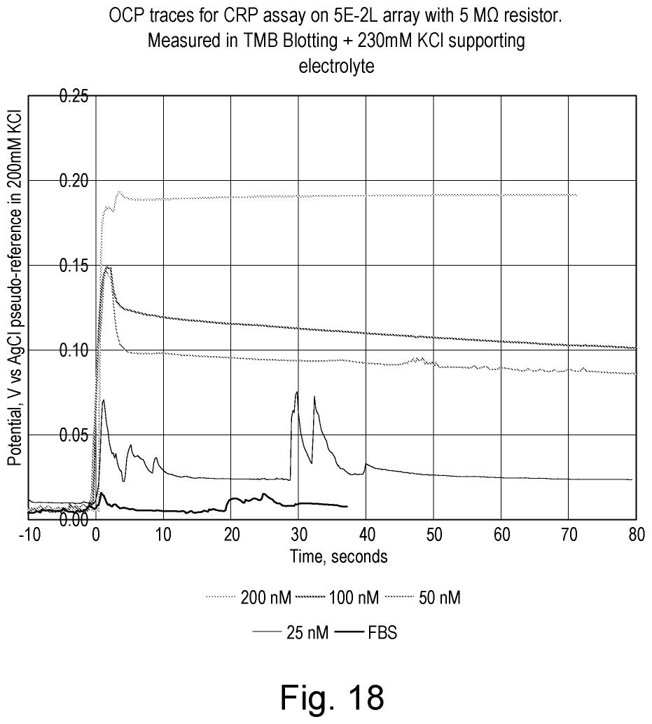

The calibration curve is shown in FIG. 17 with comparison to the curve obtained without KCl, and the time-corrected traces in FIG. 18. Internal RE was used. Small anomalies in the traces for 25 nM and FBS originate from further injections of the chromogen to ensure full exchange of reagents had occurred. It can be noted that in both cases, the signal returned to its previous steady state.

### Measurement of Multiple Electrodes Sequentially

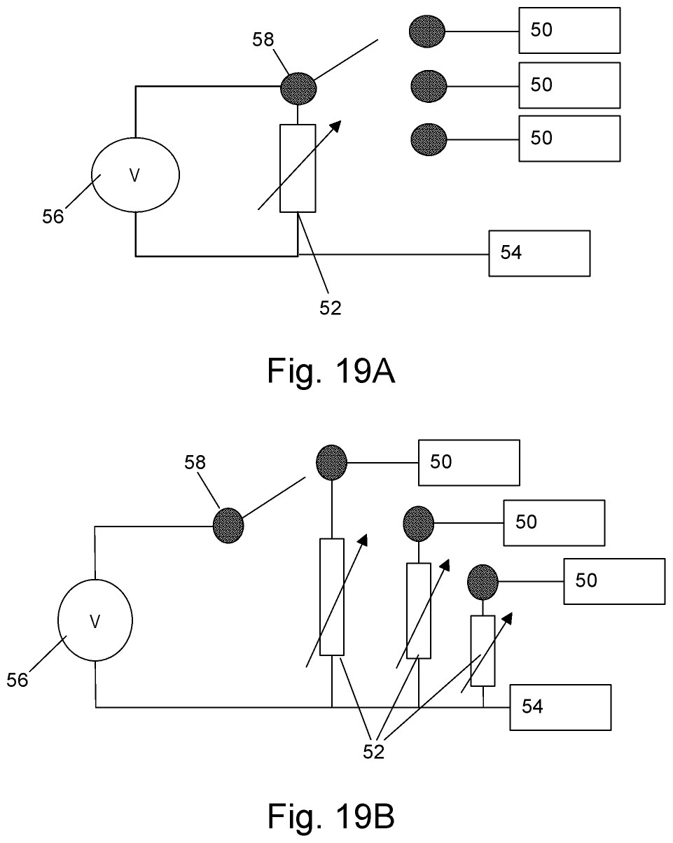

There are two main ways that multiplexing can be accomplished as illustrated in FIGS. 19A and 19B. FIGS. 19A and 19B illustrates a plurality of working electrode 50, a reference electrode 54 connectable to a voltage source 56, where a variable resistor 52 is provided between the working electrodes 50 and the voltage source 56. A switch 58 is also provided as shown in FIGS. 19A and 19B.

The first method as illustrated in FIG. 19A switches 58 between each working electrode (WE) 50 in turn. The advantage is that the working electrodes are completely separated from one another, so no current can leak between them. It may be required that the working electrode 50 may need to equilibrate with the resistor 52 only at the point of measurement, which can take time. However, the method as illustrated in FIG. 19A leads to reproducible results across the array. FIG. 19B shows sequential measurements from E1 to E5, where switching 58 occurs once steady state has been reached. The longer the duration before an electrode was connected, the longer it took to reach a steady state (as would be expected from the accumulation of precipitate without current drain). Thus, this method as illustrated in FIG. 19B can provide an alternative method of measurement and it can be used to produce results across the array.

The invention may also be understood by reference to the following clauses;

1. A method for detecting the concentration of a chemical or biological target within a sample, the method comprising the steps of:

providing an electrochemical cell with a first electrode module and a second electrode;

providing an electronic component between the first electrode module and the second electrode;

introducing the sample into the electrochemical cell;

measuring the potential difference between the first electrode module and second electrode;

converting the measured potential difference into a concentration of the chemical or biological target.

2. The method according to clause 1, further comprising the step of varying the resistance of the electronic component.

3. The method according to clause 1 or clause 2, further comprising the step of functionalising the second electrode with a biological recognition element such as an antibody.

4. The method according to any one of clauses 1 to 3, wherein the chemical or biological target is a redox poising species.

5. The method according to any one of clauses 1 to 4, further comprising the step of providing a component configured to bind to the first electrode module to generate or change the concentration of the chemical or biological target on the first electrode module.

6. A device for detecting the concentration of a chemical or biological target within a sample, the device comprising:

an electrochemical cell comprising a first electrode module and a second electrode both immersed in an electrolyte solution;

an electronic component provided between the first electrode module and the second electrode;

an electrometer configured to measure the potential difference between the first electrode module and the second electrode;

a memory configured to store data to provide conversion between measured potential difference and number of moles of chemical or biological target; and

a processor configured to identify the relevant data corresponding to the measured potential difference to process the concentration of the chemical or biological target within the sample.

7. The device according to clause 6, wherein the electronic component is a variable resistor including either a rheostat or varistor or a switched group of resistors.

8. The device according to clause 7, wherein the memory further comprises data for different values of the resistance of the electronic component.

9. The device according to any one of clauses 6 to 8, wherein the second electrode is functionalised with a biological recognition element.

10. The device according to clause 9, wherein the biological recognition element is an antibody.

11. The device according to any one of clauses 6 to 9, wherein the second electrode is made from gold.

12. The device according to any one of clauses 6 to 11, wherein the first electrode module is provided with a component configured to generate or change the concentration of the chemical or biological target.

13. The device according to clause 12, wherein the first electrode module comprising the component is configured to generate a different concentration of the chemical or biological target from the concentration of the chemical or biological target on the second electrode.

14. The device according to any one of clauses 6 to 13, wherein the first electrode module comprises a single first electrode.

15. The device according to clause 14, wherein the first electrode is made from silver.

16. The device according to clause 15, wherein the first electrode is coated in silver chloride.

17. The device according to any one of clauses 6 to 13, wherein the first electrode module comprises two electrodes, one connected to the electrometer and one connected to the electronic component.

The invention may also be further understood by reference to the following clauses;

1. A method for detecting the rate of production or depletion of a chemical or biological target within a sample, the method comprising the steps of:

providing an electrochemical cell with a first electrode module and a second electrode;

providing an electronic component between the first electrode module and the second electrode;

introducing the sample into the electrochemical cell;

repeatedly measuring the potential difference between the first electrode module and second electrode;

processing the potential difference measurements to identify a rate of change of potential difference and converting that rate of change of potential difference into a rate of production or depletion of the chemical or biological target.

2. The method according to clause 1, further comprising the step of varying the resistance of the electronic component.

3. The method according to clause 1 or clause 2, further comprising the step of functionalising the second electrode with a biological recognition element such as an antibody.

4. The method according to any one of clauses 1 to 3, wherein the chemical or biological target is a redox poising species.

5. The method according to any one of clauses 1 to 4, further comprising the step of providing a component configured to bind to the first electrode module to generate or change the concentration of the chemical or biological target on the first electrode module.

6. A device for detecting the rate of production or depletion of a chemical or biological target within a sample, the device comprising:

an electrochemical cell comprising a first electrode module and a second electrode both immersed in an electrolyte solution;

an electronic component provided between the first electrode module and the second electrode;

an electrometer configured to measure the potential difference between the first electrode module and the second electrode;

a memory configured to store data to provide conversion between measured rate of change of potential difference and rate of production or depletion of the chemical or biological target; and

a processor configured to identify the relevant data corresponding to the measured rate of chance of potential difference to process the rate of production or depletion of the chemical or biological target within the sample.

7. The device according to clause 6, wherein the electronic component is a variable resistor including either a rheostat or varistor or a switched group of resistors.

8. The device according to clause 7, wherein the memory further comprises data for different values of the resistance of the electronic component.

9. The device according to any one of clauses 6 to 8, wherein the second electrode is functionalised with a biological recognition element.

10. The device according to clause 9, wherein the biological recognition element is an antibody.

11. The device according to any one of clauses 6 to 10, wherein the second electrode is made from gold.

12. The device according to any one of clauses 6 to 11, wherein the first electrode module is provided with a component configured to generate or change the concentration of the chemical or biological target.

13. The device according to clause 12, wherein the first electrode module comprising the component is configured to generate a different concentration of the chemical or biological target from the concentration of the chemical or biological target on the second electrode.

14. The device according to any one of clauses 6 to 13, wherein the first electrode module comprises a single first electrode.

15. The device according to clause 14, wherein the first electrode is made from silver.

16. The device according to clause 15, wherein the first electrode is coated in silver chloride.

17. The device according to any one of clauses 6 to 13, wherein the first electrode module

comprises two electrodes, one connected to the electrometer and one connected to the electronic component.

Various further aspects and embodiments of the present invention will be apparent to those skilled in the art in view of the present disclosure. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A method for detecting the presence or absence of a chemical or biological target within a sample, the method comprising the steps of:
providing an electrochemical cell with a first electrode module and a second electrode;
providing an electronic component between the first electrode module and the second electrode;
functionalising the second electrode with an antibody;
introducing the sample into the electrochemical cell;
measuring the potential difference between the first electrode module and second electrode; and
confirming the presence of the chemical or biological target if the measured potential difference exceeds a predetermined threshold value.

2. The method according to claim 1, further comprising the step of:
varying the resistance of the electronic component.

3. The method according to any one of claims 1 to 2, wherein the chemical or biological target is a redox poising species.

4. The method according to any one of claims 1 to 3, further comprising the step of:
providing a component configured to bind to the first electrode module to generate or change the concentration of the chemical or biological target on the first electrode module.

5. A device for detecting the presence or absence of a chemical or biological target within a sample, the device comprising:
an electrochemical cell comprising a first electrode module, a second electrode and an electrolyte container configured to immerse both the first electrode module and the second electrode in an electrolyte solution, wherein the second electrode is functionalised with an antibody;
an electronic component provided between the first electrode module and the second electrode;
an electrometer configured to measure the potential difference between the first electrode module and the second electrode;
a memory configured to store predetermined threshold data; and
a processor configured to compare the measures potential difference and the stored threshold data to confirm the presence or absence of the chemical or biological target within the sample.

6. The device according to claim 5, wherein the electronic component is a variable resistor including either a rheostat or varistor or a switched group of resistors.

7. The device according to claim 6, wherein the memory further comprises data for different values of the resistance of the electronic component.

8. The device according to any one of claims 5 to 7, wherein the second electrode is functionalised with a biological recognition element.

9. The device according to any one of claims 5 to 8, wherein the second electrode is made from gold.

10. The device according to any one of claims 5 to 9, wherein the first electrode module is provided with a component configured to generate or change the concentration of the chemical or biological target.

11. The device according to claim 10, wherein the first electrode module comprising the component is configured to generate a different concentration of the chemical or biological target from the concentration of the chemical or biological target on the second electrode.

12. The device according to any one of claims 5 to 11 wherein the first electrode module comprises a single first electrode.

13. The device according to claim 12, wherein the first electrode is made from silver.

14. The device according to claim 12, wherein the first electrode is coated in silver chloride.

15. The device according to any one of claims 5 to 11, wherein the first electrode module comprises two electrodes, one connected to the electrometer and one connected to the electronic component.

16. A liquid handling cartridge for detecting the presence or absence of a chemical or biological target within a sample, the cartridge comprising one or more measurement chambers comprising a device according to claims 5 to 15 for performing measurements on the sample.

17. The liquid handling cartridge of claim 16 further comprising:
a main chamber;
a sample chamber for receiving a sample;
a variable pressure source conduit for connecting the main chamber to a variable pressure source;
a sample chamber conduit which fluidically connects the sample chamber to the main chamber;
a sample chamber conduit valve for opening and closing the sample chamber conduit;
a respective measurement chamber conduit for each measurement chamber, wherein each respective measurement chamber conduit fluidically connects the respective measurement chamber to the main chamber; and
a respective measurement chamber conduit valve for opening and closing each respective measurement chamber conduit.
